(19) United States
(12) Patent Application Publication
(10) Pub. No.: US 2004/0067952 A1
(43) Pub. Date: Apr. 8, 2004

(54) Method and composition for treatment of cancer

(76) Inventors: David Lawson Morris, Lugarno NSW (AU); Mohammad Hossein Pourgholami, Penshurst (AU)

Correspondence Address: Nixon & Vanderhye, 8th Floor, 1100 North Glebe Road, Arlington, VA, 22201-4714, US

(21) Appl. No.: 10/467,618

(22) PCT Filed: Jul. 16, 2002
(86) PCT No.: PCT/AU02/00954

(30) Foreign Application Priority Data
Jul. 17, 2001 (AU) PR 6443

(51) Int. Cl. A61K 31/498

(52) U.S. Cl. 514/250

(57) ABSTRACT

The present invention provides a method of treatment of tumours. The method comprises regional delivery to the site of the tumour a composition comprising a therapeutically effective amount of a riminophenazine compound. The present invention also provides composition comprising riminophenazine compounds in combination with lipids.

### FIELD OF THE INVENTION

The present invention relates to a method for the treatment of cancer and in particular, with a method for the treatment of liver cancer. The present invention further relates to compositions for use in such treatment.

### BACKGROUND OF THE INVENTION

Hepatocellular cancer (HCC) is one of the most lethal malignancies and ranks world wide as the seventh most common cancer. There is considerable variation in its incidence with it being the most common in Asian countries. Due to an insidious onset, the majority of the HC(s are unresectable at diagnosis. Current chemotherapeutic agents used systemically in the treatment of HCCs have not been very successful (1-5). In recent years advantages for regional delivery of drugs in HCC has been described. This route of administration, while, sparing the rest of the body from toxic effects, allows for the achievement of higher concentrations of the drug directly at the tumor site. Furthermore, delivery of the drug in an oil such as lipiodol, which is highly taken up and retained for long periods of time by liver tumors, would further increase the selectivity of this route of administration (6-8). This way, by achieving high local hepatic levels, hepatic tumors sensitive to clofazimine or its. analogues could be treated. However, the drug in question must be extremely soluble in oil such as lipiodol to allow for proper delivery, and later, sustained release of the drug from the lipid with in the tumor.

Clofazimine is a riminophenazine compound, with a molecular weight of 473.14 and a characteristic deep red to orange color nature under normal conditions due to its complex heterocyclic structure (9). It emerged as the most active antimycobacterial agent of a class of compound, the riminophenazine, synthesized by the laboratories of the Medical Research council of Ireland from 1944, as part of a project to find a treatment for tuberculosis. Several hundred derivatives of clofazimine have been synthesized and tested in the laboratory for potential therapeutic uses.

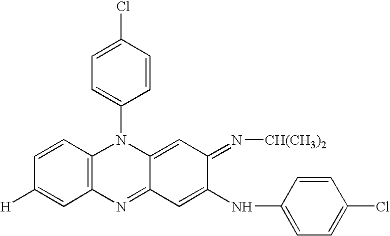

Structure of clofazimine: [C₂₇H₂₂CL₂N₂; 3-(4-chloroanilino)-10-(4-chlorophenyl)-2, 10-dihydro-2-(isopropylimino)-phenazine]

Clofazimine has been used in the treatment of mycobacterial diseases since 1962. It is clinically effective and safe in the management of a number of diseases and is mainly employed in combination with dapsone and rifampin in the treatment of multibacillary leprosy. Currently the major use of clofazimine is in the World Health Organization multiple drug therapy for lepromatous leprosy. Clofazimine is generally considered to be a safe drug. Nevertheless, there are some drawbacks and side effects associated with its use. The side effects are normally mild, dose related and reversible. The most common side effect seen is a red brown discoloration of the skin, which is visible in all patients on high doses. Certain cultures, particularly some Asian races, find the associated coloration stigmatizing and unacceptable, and this is the major cause of noncompliance in treatment regimes (9-12).

The antimicrobial effects of clofazimine or its analogues have been the subject of numerous scientific publications and patents.

Recently two groups of investigators have reported using oral clofazimine in the treatment of HCC. Ruff et al. (13) reported that, 10% (3/30) of their patients (HCC) under treatment with clofazimine, had objective response, while, 43% (13/30) had stabilization of the disease for up to 20 months. In a following study, Falkson et al. (14) reported no objective response in any of their patients under-treatment for HCC with clofazimine alone.

Van Rensburg et al. has reported the activity of clofazimine against FaDu, a human squamous cell carcinoma, PLC/PRF 5 a human HCC cell line in vitro and chemically induced sarcomas of mice and the mammary tumors of rats in vivo (15, 16). Similarly, Sri-Pathmanthan, has shown clofazimine to be active against human lung cancer cells under both in vitro and in vivo (nude mice xenografts) conditions (17). More recently U.S. Pat. No. 5,763,443 (the disclosure of which is incorporated herein by reference) has suggested multi drug resistance (MDR) activity for riminophenazines.

### SUMMARY OF THE INVENTION

The present inventors have shown clofazimine is a potent inhibitor of the proliferation of a range of liver and colorectal cancer cell lines.

Accordingly, in a first aspect, the present invention provides a method for the treatment of a tumour in a subject, the method comprising regional delivery to the site of the tumour a composition comprising a therapeutically effective amount of a compound of Formula I:

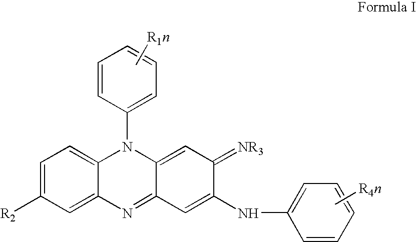

in which R₁ and R₄ are selected from the group consisting of hydrogen atoms, halogen atoms, C₁-C₃ alkyl radicals, C₁-C₃ alkoxy radicals, fluoromethoxy and trifluoromethyl radicals, R₂ is selected from the group consisting of hydrogen and halogen atoms, R₃ is selected from the group consisting of hydrogen atoms, C₁-C₄ alkyl, N,N-dialkylaminoalkyl, C₃-C₁₂ cycloalkyl, methylcyclohexyl, hydroxycyclohexyl, cycloalkylmethyl, piperidyl, alkyl substituted piperidyl and N-benzyl substituted piperidyl, and n is a number from 1 to 3 inclusive; or an analogue or metabolite thereof.

### DESCRIPTION OF FIGURES

FIG. 1: Cell proliferation in human hepatoma cell line HepG2 as measured by 3[H]thymidine incorporation and expressed as counts per minute (CPM). Cells were treated in culture for 5 days with different concentrations of clofazimine (CF, 0-5 μM). Values represent mean±s.e.m. of counts per minute (CPM) which is directly proportional to the number of viable cells present in each well at the end of the treatment period.

FIG. 2: HepG2 cells plated in 6 well plates were treated with clofazimine (0-5 mM) for 1, 3, or 7 days and viable cells remaining were counted using Trypan blue dye method. All counts were obtained in quadruplicate. Values represent mean±standard error.

FIG. 3: Rat hepatoma cells, novikoff, were grown in test tubes and treated for 1, 3 or 7 days with different concentrations of dofazimine (0-10 mM). At the end of treatment period, the number of viable cells remaining was counted using the Trypan Blue method. All counts were obtained in triplicate and the values represent mean±standard error.

FIG. 4: Inhibition of cell proliferation by clofazimine in human hepatoma cell line HepG2. Cells plated in 24 well plates, were treated in culture for 1 day with clofazimine (CF, 5 μM), lipiodol (L, 100 μl), or clofazimine-lipiodol (CF/L) and thoroughly washed. Cells were then incubated with medium alone (no drug or lipiodol added) for an extra 9 days. Cell proliferation was measured by 3[H]thymidine incorporation assay and results (mean±s.e.m.) are expressed as counts per minute (CPM).

FIG. 5: Rats (male S.D.) were inoculated in the liver with 2×10⁶ rat liver tumor cells. 7 days later, another laprotomy was performed and after measuring tumor volume (V1), through a catheter placed into the hepatic artery 100 μl of sterile normal saline, lipiodol or clofazimine (0.4 mg dissolved in 100 μl of lipiodol) was slowly infused. 7 days later, animals were euthansed, and tumor volume (V2) measured.

FIG. 6: Plasma samples from animals treated with a single intrahepatic arterial dose of clofazimine (0.4 mg in 100 μl of lipiodol) were analyzed for total bilirubin. Blood was collected through cardiac puncture, 7 days post drug treatment just prior to animal euthanasia.

FIG. 7: Rats (male S.D.) were inoculated in the liver with 2×10⁶ rat liver tumor cells. 7 days later, another laprotomy was performed and the tumor volume, (V1), measured. 24 hours later, animals were treated orally with either the vehicle [0.5% carboxymethyl cellulose (CMC)] or clofazimine (50 mg/kg in 0.5%CMC suspension) once daily for 7 days. At the end of this period and 24 hours after the last dose, animals were euthanased, and tumor volume (V2) measured.

FIG. 8: [3H]thymidine incorporation expressed as counts per minute in LOVO cells (colorectal cancer cell line) treated in culture for 5 days with different concentrations of clofazimine.

### DETAILED DESCRIPTION OF THE INVENTION

The present inventors have shown that regional administration of the riminophenazine compound to the liver provides a number of advantages in the treatment of liver tumours. The present inventors also believe that this benefit may also be obtained through regional delivery of the riminophenazine compound to tumours of other cancers such as colorectal cancer, lung cancer, breast cancer, prostate cancer, pancreatic cancer, gastric cancer, ovarian cancer, mesothelioma, renal cancer and liposarcoma.

Accordingly, in a first aspect the present invention consists in a method of treatment of a tumour in a subject, the method comprising regional delivery to the site of the tumour a composition comprising a therapeutically effective amount of a compound of Formula I:

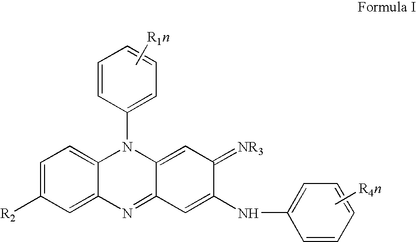

in which R₁ and R₄ are selected from the group consisting of hydrogen atoms, halogen atoms, C₁-C₃ alkyl radicals, C₁-C₃ alkoxy radicals, fluoromethoxy and trifluoromethyl radicals, R₂ is selected from the group consisting of hydrogen and halogen atoms, R₃ is selected from the group consisting of hydrogen atoms, C₁-C₄ alkyl, N,N-dialkylaminoalkyl, C₃-C₁₂ cycloalkyl, methylcydohexyl, hydroxycyclohexyl, cycloalkylmethyl, piperidyl, alkyl substituted piperidyl and N-benzyl substituted piperidyl, and n is a number from 1 to 3 inclusive; or an analogue or metabolite thereof.

Preferably R₁ substitution occurs in the 1 position and is preferably Cl.

It is preferred that n=1, R₁ is Cl, R₂ is H, R₃ is CH(CH₃)₂ and R₄ is Cl.

It is particularly preferred that riminophenazine compound is clofazimine.

The method of the present is particularly suitable for the treatment of tumor of the liver. The tumor may be a hepatoma (primary liver cancer) or a secondary cancer in the liver. Preferably regional delivery to the liver is via the hepatic artery.

The method of the present invention may also be used to treat other cancers, for example, colorectal cancer, lung cancer, breast cancer, prostate cancer, pancreatic cancer, renal cancer or secondary metastases in other organs.

Regional delivery of the riminophenazine compound may be achieved by administering the compound in a pharmaceutically acceptable formulation. The composition may be administered as continuous infusion of a solution via a pump through the major artery of the diseased organ for example hepatic artery for hepatomas. Furthermore, the composition may be administered intraperitoneally as a suspension to treat peritoneal disease arising from ovarian, pancreatic, gastric, or any other cancer.

The formulation preferably comprises a lipid. Particularly preferred are lipids for which the tumor is avid so that high concentrations of the drug may be delivered to the tumor.

It is preferred that the lipid is an oil, preferably an oil which can be imaged by an external means e.g. CT or MRI or PET. It is preferred that the oil is an iodised oil, in particular lipiodol, an iodinated ethyl ester of the poppy seed oil. In another preferred emodiment the oil is an ethyl ester of linoleic acid which may be iodinated.

While it is presently preferred that the oil is lipiodol it will be understood any oil meeting the following criteria would be suitable for regional delivery of the compound:

1) compatible with blood, and

2) suitable solvent for clofazimine or other nominated riminophenazines

It is also preferred that oil allows external monitoring of the depot. As such the oil may bear a component, chemical group or substituent which enables detection by any external means e.g. CT, MRI, PET.

Non-limiting examples of oils which may used include soybean oil (see Tibell et al, Transpl Int 1993 6:69-72), cotton seed oil, safflower oil, fatty acid monoglyceride, medium chain tryglyceride and edible oils such as olive oil, peanut oil, walnut oil, cod liver oil etc. A range chromatographically purified oils are available from Larodan Fine Chemicals AB (www.larodan.se).

As is well known all such oils (like Lipiodol) have to be used with a surfactant to be safe when injected into the circulation (see the patent of Guerbet, GB1081551).

Non-limiting examples of other lipids which may be used include nitroxyl fatty acid, NFA for use in MRI (see Gallez et al, Magn Reson Med 1993 30:592-599), polyiodinated triglycerides for CT (see Weichert et al, J Med Chem 1995 38:636-646) and polyiodinated triacylglycerols for CT (see Weichert et al, J Med Chem 1986 29:2457-65).

Compared to systemic administration, regional delivery using a lipid such as lipiodol allows achievement of higher drug concentrations in the tumour site while reducing the degree of exposure of other body organs to the unwanted effects of the drug and consequently reducing the number and the severity of side effects. In HCC this can be made even more selective and effective by choosing lipiodol as the vehicle for the drug delivery.

When injected into the hepatic artery, the oil is retained by HCCs for several weeks to over a year but is cleared from the normal liver parenchyma within 7 days. Without wishing to restrict the present invention in any way, one of the hypotheses in attempting to explain lipiodol retention in HCCs suggests that these cells are unable to clear lipiodol because they lack a reticuloendothelial kupffer cell component. The present inventors have previously shown that in vitro, vitamin D compounds such as 1, 25-dihydroxyvitamin D3 dissolved in lipiodol produce a profound and sustained inhibitory effect on HepG2 cells and when injected through the hepatic artery of tumour bearing rats, the drug is retained within the tumour (See International Patent Application Nos. PCT/AU98/00440 and PCT/AU99/00323 the disclosure of which is incorporated herein by reference).

On the basis of the present inventors experience with clofazimine, lipiodol, and hepatoma cell lines, they believe that administration of clofazimine, dissolved in an oil such as lipiodol and administered through the hepatic artery, will lead to the sustained release of the drug from the oil within the tumour cells leading to sustained inhibition of proliferation of the tumour cells.

These unique characteristics of lipiodol coupled with the potency and lipid solubility of clofazimine, makes the combination an attractive formulation for intrahepatic arterial administration in patients with HCC.

Determining the therapeutically effective amount of the riminophenazine compound can be done based on animal data using routine computational methods. Typically, the concentration of the riminophenazine compound will be at least about 0.1 μM and generally in the range of about 0.1 to about 10 μM

Clofazimine is a very lipid soluble compound with a log p value of 7.4 [(octanol/water) 9].

In a second aspect, the present invention provides a pharmaceutical composition for use in the treatment of a tumour in a subject, the composition comprising a lipid carrier and a compound of Formula I at a concentration of at least 0.1 μM:

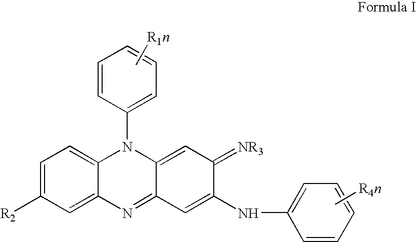

in which R₁ and R₄ are selected from the group consisting of hydrogen atoms, halogen atoms, C₁-C₃ alkyl radicals, C₁-C₃ alkoxy radicals, fluoromethoxy and trifluoromethyl radicals, R₂ is selected from the group consisting of hydrogen and halogen atoms, R₃ is selected from the group consisting of hydrogen atoms, C₁-C₄ alkyl, N,N-dialkylaminoalkyl, C₃-C₁₂ cycloalkyl, methylcyclohexyl, hydroxycyclohexyl, cycloalkylmethyl, piperidyl, alkyl substituted piperidyl and N-benzyl substituted piperidyl, and n is a number from 1 to 3 inclusive; or an analogue or metabolite thereof.

Preferably R₁ substitution occurs in the 1 position and is preferably Cl.

It is preferred that n=1, R₁ is Cl, R₂ is H, R₃ is CH(CH₃)₂ and R₄ is Cl.

It is particularly preferred that riminophenazine compound is clofazimine.

It is preferred the lipid carrier is a lipid for which the tumor is avid so that high concentrations of the drug may be delivered to the tumor.

It is preferred that the lipid is an oil, preferably an oil which can be imaged by an external means e.g. CT or MRI or PET. It is preferred that the oil is an iodised oil, in particular lipiodol, an iodinated ethyl ester of the poppy seed oil.

While it is presently preferred that the oil is lipiodol it will be understood any oil meeting the following criteria would be suitable for regional delivery of the compound:

1) compatible with blood, and

2) suitable solvent for clofazimine or other nominated riminophenazines

It is also preferred that oil allows external monitoring of the depot. As such the oil may bear a component, chemical group or substituent which enables detection by any external means e.g. CT, MRI, PET.

Non-limiting examples of oils which may used include soybean oil (see Tibell et al, Transpl Int 1993 6:69-72), fatty acid monoglyceride, medium chain tryglyceride and edible oils such as olive oil, peanut oil, walnut oil, cod liver oil etc

As is well known all such oils (like Lipiodol) have to be used with a surfactant to be safe when injected into the circulation (see the patent of Guerbet, GB1081551).

Non-limiting examples of other lipids which may be used include nitroxyl fatty acid, NFA for use in MRI (see Gallez et al, Magn Reson Med 1993 30:592-599), polyiodinated triglycerides for CT (see Weichert et al, J Med Chem 1995 38:636-646) and polyiodinated triacylglycerols for CT (see Weichert et al, J Med Chem 1986 29:2457-65).

Preferably the riminophenazine compound is present in the composition in a concentration of at least about 0.5 μM. The upper limit on the concentration of riminophenazine compound is determined by the solubility of the compound. It is preferred, however, that the concentration of the riminophenazine compound is in the range of about 0.1 to about 10 μM.

In a further aspect the present invention consists in the use of a compound of Formula I:

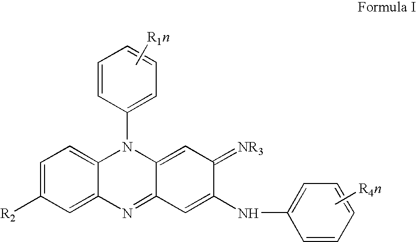

in which R₁ and R₄ are selected from the group consisting of hydrogen atoms, halogen atoms, C₁-C₃ alkyl radicals, C₁-C₃ alkoxy radicals, fluoromethoxy and trifluoromethyl radicals, R₂ is selected from the group consisting of hydrogen and halogen atoms, R₃ is selected from the group consisting of hydrogen atoms, C₁-C₄ alkyl, N,N-dialkylaminoalkyl, C₃-C₁₂ cycloalkyl, methylcyclohexyl, hydroxycyclohexyl, cydoalkylmethyl, piperidyl, alkyl substituted piperidyl and N-benzyl substituted piperidyl, and n is a number from 1 to 3 inclusive; or an analogue or metabolite thereof; in the preparation of a medicament for the treatment of tumours in a subject, the medicament being adapted adapted for regional delivery to the site of the tumour.

Preferably R₁ substitution occurs in the 1 position and is preferably Cl.

It is preferred that n=1, R₁ is Cl, R₂ is H, R₃ is CH(CH₃)₂ and R₄ is Cl.

It is particularly preferred that riminophenazine compound is clofazimine.

The medicament is particularly suitable for the treatment of tumor of the liver. The tumor may be a hepatoma (primary liver cancer) or a secondary cancer in the liver. Preferably regional delivery to the liver is via the hepatic artery.

The medicament may also be used to treat other cancers, for example, colorectal cancer, lung cancer, breast cancer, prostate cancer, pancreatic cancer, renal cancer or secondary metastases in other organs.

The medicament preferably further comprises a lipid. Particularly preferred are lipids for which the tumor is avid so that high concentrations of the drug may be delivered to the tumor.

It is preferred that the lipid is an oil, preferably an oil which can be imaged by an external means e.g. CT or MRI or PET. It is preferred that the oil is an iodised oil, in particular lipiodol, an iodinated ethyl ester of the poppy seed oil.

While it is presently preferred that the oil is lipiodol it will be understood any oil meeting the following criteria would be suitable for regional delivery of the compound and external monitoring of the depot:

1) compatible with blood

2) suitable solvent for clofazimine or other nominated riminophenazines

3) bearing a component, chemical group or substituent which enables detection by any external means e.g. CT, MRI, PET.

Non-limiting examples of oils which may used include soybean oil (see Tibell et al, Transpl Int 1993 6:69-72), cotton seed oil, safflower oil, fatty acid monoglyceride, medium chain tryglyceride and edible oils such as olive oil, peanut oil, walnut oil, cod liver oil etc. A range chromatographically purified oils are available from Larodan Fine Chemicals AB (www.larodan.se).

As is well known all such oils (like Lipiodol) have to be used with a surfactant to be safe when injected into the circulation (see the patent of Guerbet, GB1081551).

Non-limiting examples of other lipids which may be used include nitroxyl fatty acid, NFA for use in MRI (see Gallez et al, Magn Reson Med 1993 30:592-599), polyiodinated triglycerides for CT (see Weichert et al, J Med Chem 1995 38:636-646) and polyiodinated triacylglycerols for CT (see Weichert et al, J Med Chem 1986 29:2457-65).

Compared to systemic administration, regional delivery using a lipid such as lipiodol allows achievement of higher drug concentrations in the tumour site while reducing the degree of exposure of other body organs to the unwanted effects of the drug and consequently reducing the number and the severity of side effects. In HCC this can be made even more selective and effective by choosing lipiodol as the vehicle for the drug delivery.

As used herein the term “regional delivery”, and variations such as “regionally delivering”, means delivery either directly to the tumour, delivery to a vessel directly supplying the affected organ, such as the hepatic artery for liver cancer, or delivery to a body cavity proximal the tumour, such as intraperitoneally for pancreatic cancer.

As used herein the term “individual” is used in its broadest sense and is intended to cover human and non-human animals

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in the specification are herein incorporated by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

In order that the nature of the present invention may be more fully understood the invention will now be described with reference to the following non-limiting embodiments.

### EXAMPLE 1

In vitro Tests Against Liver Cancer Cells

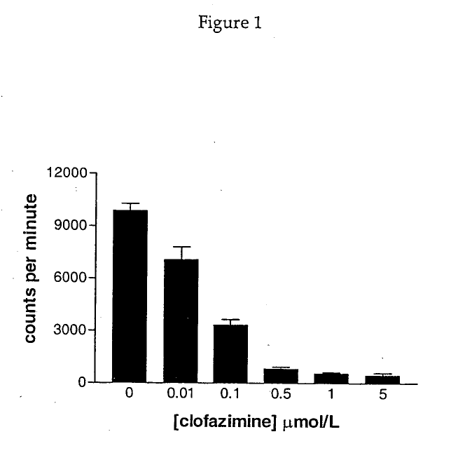

Method: [³H]Thymidine incorporation assay was employed to study the effect of clofazimine on cell proliferation. Here, adherent cells (5/000-10/000) were plated into 24-well Corning tissue culture dishes and exposed to culture medium (MEM 5% FBS) containing the vehicle or different concentrations of clofazimine (10⁻⁹ to 10⁻⁵ moles per liter). For Novikoff which is a detached rat cell line, 2000 cells were suspended in 2 ml of DMEM (5%FBS) and kept under the same condition as for attached cells. Media were replaced with fresh media on alternate days. At the end of the treatment period (5-10 days), cell cultures were assayed for thymidine incorporation by the addition of 0.5 μCi of [3H]thymiydine to each well for the last 4 h of culture. The amount of radioactivity incorporated was determined using β-scintillation counter (18). Results are presented as the actual counts per minute against the concentration of the drug used in the culture media or as % inhibition (reduction in thymidine incorporation compared to controls). Treatment of different liver cancer cell lines, HUH-7, HepG2, SKHEP-1, Hep3-B (human cell lines) and Novikoff (rat cell line) with clofazimine led to dose-dependent inhibition of [3H]thymidine incorporation by these cells (Table 1 and FIG. 1).

TABLE 1
Effect of clofazimine on the proliferation ([3H]thymidine
incorporation) of liver cancer cell lines in vitro.

| | [Clofazimine μM] | | | | |
|---|---|---|---|---|---|
| Cell line | 0.01 | 0.1 | 0.5 | 1.0 | 5.0 |
| HUH-7 | 0 | 38.3 ± 7.2 | 51.1 ± 3.4 | 88.9 ± 1.1 | 96.7 ± 0.9 |
| HepG2 | 21.0 ± 1.9 | 38.2 ± 2.2 | 52.9 ± 4.7 | 75.6 ± 2.7 | 92.4 ± 1.9 |
| SKHEP-1 | 30.3 ± 2.9 | 62.7 ± 7.1 | 69.0 ± 3.2 | 70.9 ± 2.1 | 97.1 ± 1.7 |
| Hep3-β | 28.4 ± 7.4 | 62.3 ± 3.1 | 91.6 ± 1.1 | 94.3 ± 0.07 | 95.4 ± 1.4 |
| Novikoff | 0 | 0 | 11.7 ± 3.3 | 18.9 ± 2.9 | 42.7 ± 4.1 |

Values represent mean±s.e.m of % inhibition of [3H] thymidine incorporation (compared to control) for each cell line when treated for 5 days with different concentrations of clofazimine.

These results show that, the human cell lines are quite susceptible to the antiproliferative effects of dofazimine while, the rat cell line Novikoff which generally is a very resistant cell line to chemotherapy and radiotherapy (19) shows modest susceptibility at higher drug concentrations. Amongst the human liver cancer cell lines tested, Hep3-β exhibited the highest degree of susceptibility to the antiproliferative effects of clofazimine (FIG. 1).

### EXAMPLE 2

In vitro Test Against HepG2 Cells

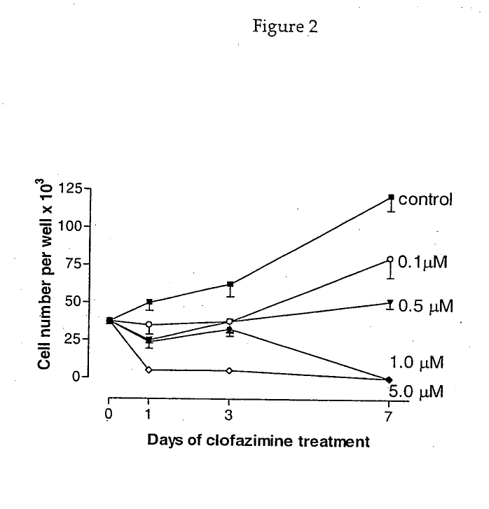

HepG2 cells plated in 6 well plates were treated with clofazimine (0-5 μM) for 1, 3, or 7 days and viable cells remaining were counted using Trypan blue dye method. All counts were obtained in quadruplicate. The results are shown in FIG. 2 where the values represent mean±standard error.

### EXAMPLE 3

In vitro Test Against Rat Hepatoma Cells, Novikoff

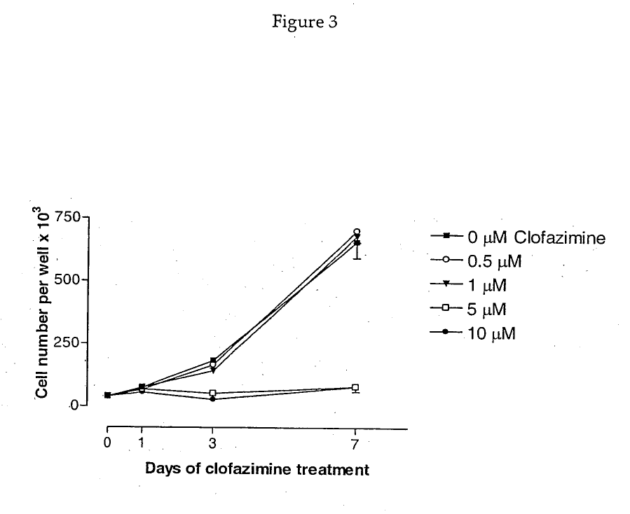

Rat hepatoma cells, novikoff, were grown in test tubes and treated for 1, 3 or 7 days with different concentrations of clofazimine (0-10 μM). At the end of treatment period, the number of viable cells remaining was counted using the Trypan Blue method. All counts were obtained in triplicate and the values represent mean±standard error. The results are shown in FIG. 3.

### EXAMPLE 4

In vitro Test Against Liver Cancer Cells Using Lipiodol

It has been shown that, certain oils and lipiodol in particular are taken up and retained by liver cancer cells. In this respect we have shown that:

under cell culture conditions, lipiodol is highly taken up by liver cancer cells (18)

in rats bearing liver tumors, lipiodol is taken up and retained with in the tumor (19)

in patients with liver tumors, administration of large doses of 1,25-dihydroxyvitamin D3 dissolved in lipiodol and infused through the hepatic artery, does not lead to the development of hypercalcemia

clofazimine is highly soluble and stable in lipiodol

To investigate if dofazimine dissolved in lipiodol is taken up and released gradually with in the cell to produce a sustained antiproliferative effect, the following experiment was carried out.

Subconfluent HepG2 cells were plated in 24 well tissue culture plates at 10/000 cells per well and incubated for 24 h in an incubator at 37° C. with humidified 5% CO₂ atmosphere. The medium was then replaced with one containing a 5 μM concentration of clofazimine prepared in either MEM or MEM plus lipiodol (0.5% v/v). To do this, clofazimine dissolved in ethanol, was placed in the test tube, the ethanol evaporated under a stream of nitrogen gas, and the drug recovered by the addition of lipiodol and finally reconstituted in medium to give the desired concentrations of the drug and lipiodol. Two groups of control cells were treated either with clofazimine made up in the medium (no lipiodol) or with lipiodol containing medium (no drug). 24 hours later, for all cells, the medium was replaced with normal medium containing neither drug nor lipiodol. From here onwards, media was replaced on alternate days, for the following 9 days. At the end of the treatment period, cells were assayed for thymidine incorporation (as described above).

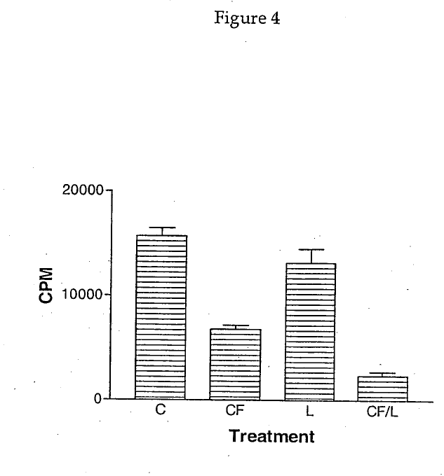

Results obtained (FIG. 4) reveal that, brief treatment of HepG2 cells with clofazimine dissolved in lipiodol and diluted in cell culture media (0.5% V/V), results in sustained inhibition of proliferation of the cells, long after (9 days) the removal of the drug from the cell culture media. This is probably due to the uptake of the oil by the cells followed by the sustained release of the drug from it with in the cell.

From these in-vitro results it may be assumed that, lipiodol taken up by the cells act as drug depots leading to the continuous exposure of the cells to dofazimine, long after the removal of the drug from the medium. Consequently, proliferation and hence [3H]thymidine incorporation is significantly (p<0.01) reduced in cells exposed to the dofazimine /lipiodol treatment.

### EXAMPLE 5

In vivo Tests in Novikoff Tumor Bearing Rats

To investigate whether dofazimine dissolved in lipiodol is active in vivo, the novikoff rat tumor model was employed. Here, the following procedure was carried out. Rats were given a general anesthetic (halothane gas) and a lapratomy performed. Then 2×10⁶ novikoff cells suspended in 100 μL of medium were instilled beneath the liver capsule using a 26G 3/8 tuberculin needle. The abdominal incision was closed with sutures. All procedures were carried out under general anesthesia and sterile conditions.

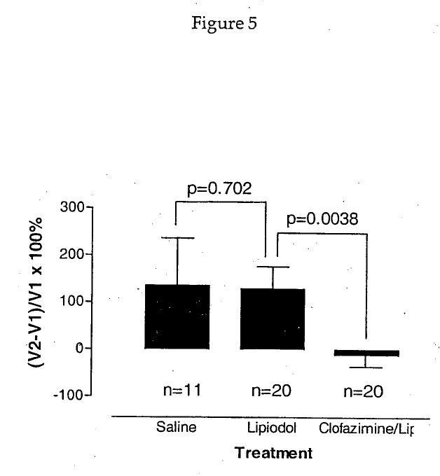

Seven days later, another lapratomy was performed and after measuring tumor volume (V1), through a catheter placed into the hepatic artery 100 μl of sterile normal saline, lipiodol or clofazimine (0.4 mg dissolved in 100 μl of lipiodol) was slowly infused. 7 days later, animals were euthansed, and tumor volume (V2) measured. The results are shown in FIG. 5.

### EXAMPLE 6

Bilirubin Measurement

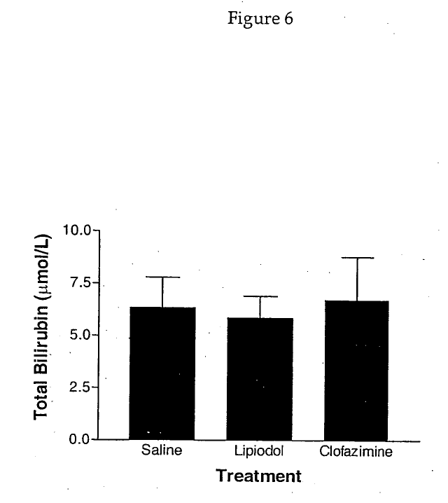

Plasma samples from animals treated with a single intrahepatic arterial dose of clofazimine (0.4 mg in 100 μl of lipiodol) were analyzed for total bilirubin. Blood was collected through cardiac puncture, 7 days post drug treatment just prior to animal euthanasia. The results are shown in FIG. 6.

### EXAMPLE 7

Oral Administration of Clofazimine

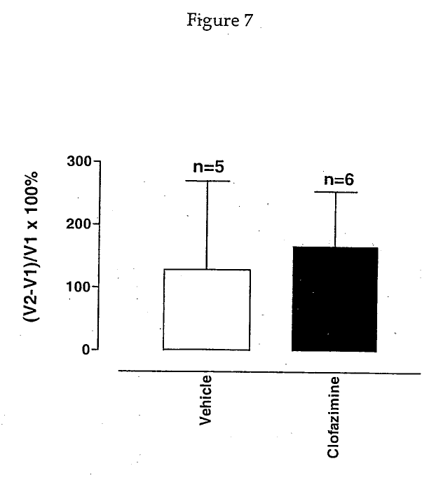

Rats (male S.D.) were inoculated in the liver with 2×10⁶ rat liver tumor cells. 7 days later, another laprotomy was performed and the tumor volume (V1), measured. 24 hours later, animals were treated orally with either the vehicle [0.5% carboxymethyl cellulose (CMC)] or dofazimine (50 mg/kg in 0.5% CMC suspension) once daily for 7 days. At the end of this period and 24 hours after the last dose, animals were euthanased, and tumor volume (V2) measured. The results are shown in FIG. 7.

### EXAMPLE 8

In vitro Tests Against Human Colorectal Cancer Cells

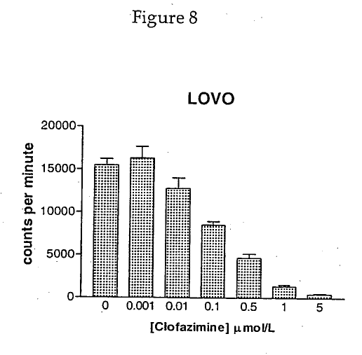

We have also shown for the first time, that, in vitro, treatment of colorectal cell lines C-170, HT-29 and LOVO with clofazimine, leads to profound inhibition of proliferation of these cells as measured by [3H]thymidine incorporation (FIG. 8). Other colorectal cancer cells (HT-29 and C-170) were also suppressed in vitro in a similar manner to LOVO where, over a 5 day treatment period, 1 μM concentrations of clofazimine produced >70% inhibition of cell proliferation in these 2 cell lines as well.

These results show that, rats treated with clofazimine dissolved in lipiodol have substantially smaller tumors than lipiodol or saline treated animals. This is in agreement with the above described in vitro results showing that, clofazimine-lipiodol is probably originally taken up and stored in the tumor cells and then released to produce a pharmacologically effective concentration in the tumor vicinity leading to inhibition of tumor growth. Furthermore, the ability to inhibit in vivo proliferation of novikoff cells is quite interesting in that, novikoff is generally a resistant cell line and was the least sensitive cell line to clofazimine in the in-vitro studies (Table 1).

Based on these results it is believed that, in patients with tumours, in particular liver cancer, clofazimine dissolved in lipiodol and administered regionally, will be taken up by tumor cells causing high local concentrations of the drug and leading to efficacy, while, at the same time, sparing rest of the body from undesirable exposure to high concentrations of the drug.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### References

1 Okada, S. Chemotherapy in hepatocellular carcinoma. Hepatogastroenterology 45: 1259-63, 1998.

2 Tang, Z. Y. Hepatocellular carcinoma. J. Gastroenterol. Hepatol. 15: (Suppl.) G1-7, 2000.

3 Akriviadis, E. A., Llovet, J. M., Efremidis, S. C. Hepatocellular carcinoma. Br. J. Surg. 85: 1319-31, 1998.

4 Mathurin, P., Rixe, D., Carbonell, N. et al. Review article: Overview of medical treatments in unresectable hepatocellular carcinoma—an impossible meta analysis? Aliment. Pharmacol. Ther. 12: 111-26, 1998.

5 Farmers, D. G., Rosove, M. H., Shaked, A. et al. Current treatment modalities for hepatocellular carcinoma. Ann. Surg. 219: 236-47, 1994.

6 Bhattacharya, S., Dhillon A. P., Winslet M. C., et al. Human liver cancer cells and endothelial cells incorporate iodized oil. Br. J. Cancer 73: 877-881, 1996.

7 Nakakuma, K., Tashiro, S., Hiraoka, T., et al. Studies on anticancer treatment with an oily anticancer drug injected into the ligated feeding hepatic artery for liver cancer. Cancer 52: 2193-2200, 1983.

8 Chou, F. I., Fang, K .C., Chung, C., et al. Lipiodol uptake and retention by human hepatoma cells. Nucl. Med. Biol. 22: 379-386, 1995.

9 O'Conner, R., O'Sullivan, J. F., O'Kennedy, R. The pharmacology, metabolism and chemistry of clofazimine. Drug Metab. Rev. 27: 591-614, 1995.

10 Arbiser, J. J., Moschella, S. L. M. Clofazimine: A review of its medical uses and mechanisms of action. J. A. Acad. Dermatol. 32: 241-7, 1995.

11 Dollery, C. (ed.) Clofazimine. In “Therapeutic drugs” .pp C282-C286, Churchill-Livingstone, 1995.

12 Reddy, V. M., Nadadhur, G., Daneluzzi, D., et al. Antituberculosis activities of clofazimine and its new analogs B4154 and B4157. Antimicrob. Agents Chemother. 40: 633-636, 1996.

13 uff, P., Chasen, M. R., Long, J. E. H. et al. A phase II study of oral clofazimine in unresectable and metastatic hepatocellular carcinoma. Ann. Oncol. 9: 217-219, 1998.

14 Falkson, C. I., Falkson, G. A phase II evaluation of clofazimine plus doxorubicin in advanced, unresectable primary hepatocellular carcinoma. Oncology 57: 232-235, 1999.

15 Van Rensburg, C. E. J., Durandt, C., Garlinski, P. J. et al. Evaluation of the antineoplastic activities of the riminophenazine agents clofazimine and B669 in tumor-bearing rats and mice. Int. J. Oncol. 3: 1011-1013, 1993.

16 Van Rensburg, C., E., J., Van Staden, A., M., Anderson, R. The riminophenazine agents clofazimine and B669, inhibit the proliferation of cancer cell lines in vitro by phospholipase A₂-mediated oxidative and non-oxidative mechanisms. Cancer Res. 53: 318-323, 1993.

17 Sri-Pathinanathan, R., M., Plumb, J., A., Fearon, K., C., H. Clofazimine alters the energy metabolism and inhibits the growth rate of human lung-cancer cell line in vitro and in vivo. Int. J. Cancer 56: 900-905, 1994.

18 Labarca, C., Paigen K. A simple, rapid, and sensitive DNA assay procedure. Anal. Biochem. 102: 344-352, 1980.

19 Hafeli, U. O., Casillas, S., Dietz, D. W. et al. Hepatic tumor radioembolization in a rat model using radioactive rhenium (¹⁸⁶RE/¹⁸⁶RE) glassmicrospheres. Int. J. Rad. Oncol. Biol. Phys. 44: 189-199, 1999.

20 Kurth, S., Bulian, D., Kreft, B. et al. Intraarterial hepatic chemotherapy with fluorouracil, fluorodeoxyuridine, motomycin C, cisplatin or methotrexate as single-agent anticancer drugs for transplanted experimental liver tumor in rats. J. Cancer Res. Clin. Oncol. 122: 421-426, 1996.

21 Pourgholarni, M. H., Akhter, J., Finaly, I. G. et al. 1,25-Dihydroxyvitamin D3 dissolved in lipiodol produces a sustained antiproliferative effect in the human hepatoblastoma cell line HepG2. Anticancer Res. 20: 723-728, 2000.

22 Finlay, I. G., Stewart, G. J., Pourgholami, M. H. et al. The use of lipiodol and medium chain triglycerides as delivery agents for hepatic arterial administration of 1,25 dihydroxyvitarnin D3-A potential new treatment for hepatoma. Anticancer Res. 20: 2705-2710, 2000.

1. A method of treatment of a tumour in a subject, the method comprising regional delivery to the site of the tumour a composition comprising a therapeutically effective amount of a compound of Formula I: 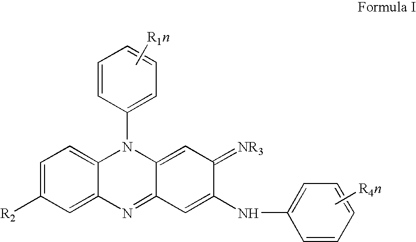
in which R₁ and R₄ are selected from the group consisting of hydrogen atoms, halogen atoms, C₁-C₃ alkyl radicals, C₁-C₃ alkoxy radicals, fluoromethoxy and trifluoromethyl radicals, R₂ is selected from the group consisting of hydrogen and halogen atoms, R₃ is selected from the group consisting of hydrogen atoms, C₁-C₄ alkyl, N,N-dialkylaminoalkyl, C₃-C₁₂ cycloalkyl, methylcyclohexyl, hydroxycyclohexyl, cydoalkylmethyl, piperidyl, alkyl substituted piperidyl and N-benzyl substituted piperidyl, and n is a number from 1 to 3 inclusive; or an analogue or metabolite thereof.

2. A method according to claim 1 wherein the R₁ substitution occurs in the 1 position and is preferably Cl.

3. A method according to claim 1 or claim 2 wherein n=1, R₁ is Cl, R₂ is H, R₃ is CH(CH₃)₂ and R₄ is Cl.

4. A method according to any one of claims 1 to 3 wherein the riminophenazine compound is clofazimine.

5. A method according to any one of claims 1 to 4 wherein the tumour is hepatoma or a secondary cancer in the liver.

6. A method according to any one of claims 1 to 5 wherein the regional delivery is via the hepatic artery.

7. A method according to any one of claims 1 to 4 wherein the tumour is selected from the group consisting of colorectal cancer, lung cancer, breast cancer, prostate cancer, pancreatic cancer, renal cancer and secondary metastases in other organs.

8. A method according to any one of claims 1 to 7 wherein the composition is administered as continuous infusion of a solution via a pump through a major artery.

9. A method according to any one of claims 1 to 7 wherein the composition is administered intraperitoneally.

10. A method according to any one of claims 1 to 9 wherein the composition further comprises a lipid.

11. A method according to claim 10 wherein the lipid is a lipid for which the tumor is avid.

12. A method according to claim 10 or 11 the lipid is an oil, preferably an oil which can be imaged by an external means e.g. CT or MRI or PET.

13. A method as claimed in claim 11 wherein the oil is an iodised oil.

14. A method as claimed in claim 11 wherein the oil is lipiodol.

15. A method according to claim 10 wherein the lipid is selected from the group consisting of soybean oil, fatty acid monoglyceride, medium chain tryglyceride, olive oil, peanut oil, walnut oil, cod liver oil, nitroxyl fatty acid, ethyl linoleate, polyiodinated triglycerides and polyiodinated triacylglycerols.

16. A pharmaceutical composition for use in the treatment of a tumour in a subject, the composition comprising a lipid carrier and a compound of Formula I at a concentration of at least 0.1 μM: 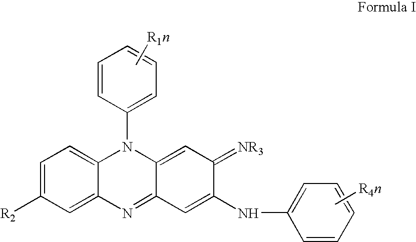
in which R₁ and R₄ are selected from the group consisting of hydrogen atoms, halogen atoms, C₁-C₃ alkyl radicals, C₁-C₃ alkoxy radicals, fluoromethoxy and trifluoromethyl radicals, R₂ is selected from the group consisting of hydrogen and halogen atoms, R₃ is selected from the group consisting of hydrogen and halogen atoms, R₃ is selected from the group consisting of hydrogen atoms, C₁-C₄ alkyl, N,N-dialkylaminoalkyl, C₃-C₁₂ cycloalkyl, methylcydohexyl, hydroxycyclohexyl, cycloalkylmethyl, piperidyl, alkyl substituted piperidyl and N-benzyl substituted piperidyl, and n is a number from 1 to 3 inclusive; or an analogue or metabolite thereof wherein the lipid carrier is selected from the group consisting of an iodised oil, soybean oil, fatty acid monoglyceride, medium chain tryglyceride, olive oil, peanut oil, walnut oil, cod liver oil, mitroxyl fatty acid, ethyl linoleate, polyiodinated triglycerides and polyiodinated triacylglycerols.

17. A composition according to claim 16 wherein the R₁ substitution occurs in the 1 position and is preferably Cl.

18. A composition according to claim 16 or 17 wherein n=1, R₁ is Cl, R₂ is H, R₃ is CH(CH₃)₂ and R₄ is Cl.

19. A composition according to any one of claims 16 to 18 wherein the riminophenazine compound is clofazimine.

20. A composition according to any one of claims 16 to 19 which the iodised oil is lipiodol.

21. A composition according to any one of claims 16 to 20 wherein the riminophenazine compound is present in the composition in a concentration of at least about 0.5 μM.

22. A composition according to any one of claims 16 to 21 wherein the concentration of the riminophenazine compound is in the range of about 0.1 to about 10 μM.
